(19) United States
(12) Patent Application Publication
(10) Pub. No.: US 2021/0145869 A1
(43) Pub. Date: May 20, 2021

(54) MICRONUTRIENT SUPPLEMENTS

(71) Applicant: Gregory L. Hall, Mayfield Village, OH (US)

(72) Inventor: Gregory L. Hall, Mayfield Village, OH (US)

(21) Appl. No.: 16/951,996

(22) Filed: Nov. 18, 2020

Related U.S. Application Data
(60) Provisional application No. 62/937,656, filed on Nov. 19, 2019.

(51) Int. Cl. A61K 33/34; A61K 31/519; A61K 31/525; A61K 31/355; A61K 31/714; A61K 31/375; A61K 31/593; A61K 31/4415; A61K 31/122; A61K 33/06; A61K 33/32; A61K 33/00; A61K 31/14; A61K 31/01; A61K 33/30; A61K 33/26; A61K 33/04; A61K 31/015; A61K 31/455; A23L 33/00; A23L 33/155; A23L 33/16

(52) U.S. Cl. CPC A61K 33/34; A23V 2002/00; A61K 31/525; A61K 31/355; A61K 31/714; A61K 31/375; A61K 31/593; A61K 31/4415; A61K 31/122; A61K 33/06; A61K 33/32; A61K 33/00; A61K 31/14; A61K 31/01; A61K 33/30; A61K 33/26; A61K 33/04; A61K 31/015; A61K 31/455; A23L 33/40; A23L 33/155; A23L 33/16; A61K 31/519

(57) ABSTRACT

Micronutrient supplements individualized for one or more of sex, age, height and weight, pregnancy/lactation status, race/ethnicity, living environment, family income, family size, educational status and achievement, personal health history, family health history, eating status, and dietary inventory are detailed. Specific micronutrient supplements for populations of a particular race and ethnicity are disclosed. Methods of formulating and use the micronutrient supplements are further disclosed.

### CROSS-REFERENCE TO RELATED APPLICATIONS

The present application claims the priority benefit of U.S. provisional patent application Ser. No. 62/937,656, entitled MICRONUTRIENT SUPPLEMENTS, filed Nov. 19, 2019, hereby incorporated herein by reference in its respective entirety.

### TECHNICAL FIELD

The present disclosure generally relates to micronutrient supplements and more specifically to micronutrient supplements that are tailored to meet the micronutrient needs of specific individuals or groups.

### BACKGROUND

Micronutrient supplements, sometimes referred to as vitamin or mineral dietary supplements, refer to supplements which contain one or more essential nutrients that cannot by synthesized by the body and which are required for proper metabolism. When not provided for in a micronutrient supplement, individuals obtain essential nutrients through dietary consumption and from exposure to their environment (e.g., sunlight for Vitamin D). Administering of micronutrient supplements overcomes the risk of malnutrition inherent when individuals have dietary intakes and/or routines that do not provide appropriate quantities of the required micronutrients.

### BRIEF DESCRIPTION OF THE DRAWINGS

FIG. 1 depicts a graph showing the dietary reference intakes for various life stage groups as published by the Food and Nutrition Board, Institute of Medicine, National Academies.

FIG. 2 depicts a graph showing the relative differences in mean serum vitamin levels between females and males as published by the National Center for Environmental Health at the Center for Disease Control (Atlanta, Ga.).

FIG. 3 depicts a graph showing the incidence of thyroid cancer by gender and ethnicity.

FIG. 4 depicts a graph showing the relative differences in mean serum vitamin levels between African Americans and white Americans of European descent as published by the National Center for Environmental Health at the Center for Disease Control (Atlanta, Ga.).

FIG. 5 depicts a graph showing food consumption at home by race/ethnicity as published by the United States Department of Agriculture Economic Research Service.

FIG. 6 depicts a graph showing the age-adjusted death rates for the top five cause of cancer death, by race and Hispanic ethnicity for the United States in 2014.

FIG. 7 depicts a graph showing the prevalence of cognitive impairment among Americans aged 55 and older by age and race/ethnicity.

FIG. 8 depicts a graph showing the number of new cases of end stage renal disease due to diabetes, by ethnicity between 2003 and 2013.

FIG. 9 depicts a graph showing the U.S. population-wide nutrient ranges.

### DETAILED DESCRIPTION

The present disclosure generally relates to micronutrient supplements that are congruent with the genetic/epigenetic needs, dietary patterns, environment (urban, rural, tropical, impoverished, affluent, etc.) and population outcomes consistent with an individual's self-identified race/ethnicity, and reported personal/family medical history. The micronutrient supplements described herein are “individualized” based on research-verified outcomes built on one or more of these self-identified variables. For example, certain micronutrient supplements are described for particular races and ethnicities. As used herein, races and ethnicities refer to the definitions published by the National Institute of Health in Notice NOT-OD-15-089 unless otherwise specified. The racial and ethnic categories detailed therein include “American Indian or Alaska Native”; “Asian”; “Black or African American”; “Hispanic or Latino”; “Native Hawaiian or Other Pacific Islander”; and “White”.

It has been unexpectedly discovered that micronutrient supplements can be improved by reducing, or substantially removing, certain micronutrients from a supplement. This surprising modification improves health outcomes by appreciating that excessive micronutrient consumption can be harmful and that certain individuals may already have sufficient amounts of a particular nutrient based on their race, diet, genetics, or the like. In the case of micronutrients, there actually can be “too much of a good thing.” In certain embodiments, excluding or reduced an amount of a micronutrient can mean at least about 50% less of that micronutrient; at least about 75% less of that micronutrient; or at least about 90% less of that micronutrient when compared to the amount of that micronutrient in a conventional micronutrient supplement. As can be appreciated, a reduced amount of a nutrient may be present as a binder or filler in certain micronutrient supplements. In certain embodiments, a reduced amount of a micronutrient can alternatively mean about 25 mg or less of that micronutrient; about 15 mg or less of that micronutrient; about 10 mg of less of that micronutrient; about 5 mg or less of that micronutrient; about 2.5 mg or less of that micronutrient; or about 1 mg or less of that micronutrient. Substantially removing a micronutrient means that the micronutrient was not intentionally added to the micronutrient supplement but that the micronutrient may be present in trace quantities as, for example, a trace impurity.

The present disclosure recognizes the need to modify micronutrients based on the characteristics of an individual. For example, it has long been accepted that vitamin D levels are consistently lower in persons with significant melanin content in their skin (African Americans and East Indians, for example). Vitamin D deficiency, either through decreased sun exposure or dietary intake, has been associated with a number of poor health outcomes including cancers of the prostate, colon and breast as well as renal/kidney health and asthma stability. Similarly, lactose intolerance also has a significant genetic component with people of East Asia and Sub-Saharan ancestry having greater than 90 percent of adults unable to properly digest milk (U.S. National Library of Medicine). As can be appreciated, the nutritional benefits of dairy products (including significant contributions of calcium, vitamin D, riboflavin and vitamin B12) are lost to lactose intolerant individuals. Studies have found that African Americans frequently do not meet dietary recommendations for calcium, phosphorus and magnesium due principally to decreased dairy consumption.

Vitamin level variations based on geographic regions are also well known, most notably with sun-dependent vitamin D levels higher in equatorial regions and much lower in areas with less sun exposure.

Other vitamin deficiency patterns exist as well. For example, a study conducted at Duke University Medical Center found that in African Americans, but not white Americans, lower levels of beta-carotene and vitamin C were significantly associated with early markers implicated in cardiometabolic conditions and cancer.

Paradoxically, there are also significant risks and poor health outcomes associated with certain vitamin supplementation. Vitamin E supplementation was studied in over 130,000 people and those that took 400 IU (the most common supplement dose) or higher, had an overall higher risk of dying from any cause. Dietary supplementation with vitamin E was shown to significantly increase the risk of prostate cancer in healthy men. Vitamin A was also shown to increase prostate cancer risk in some studies but have also been shown to offer significant protective effects in preventing lung and urinary bladder cancer. β-carotene and some B vitamins have been associated with increased lung cancer.

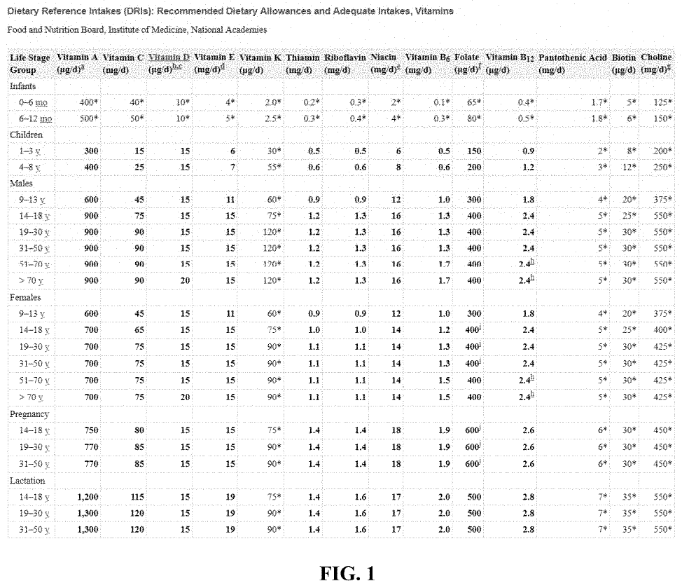

Overall, research has always shown distinct differences in micronutrient impact and requirements based on fundamental genetics. It is well accepted that men and women have distinctly different micronutrient needs with women requiring more iron during their reproductive years and more calcium with advancing age. The “recommended dietary allowance” (RDA) for both vitamins and minerals differ by sex with men generally requiring more replacement of a given vitamin or mineral than women. The RDA, which is compiled by the Food and Nutrition Board of the Institute of Medicine, is also tailored by age, pregnancy status, and lactation needs. These evidenced based variations in requirements are based on outcomes research considering lean mass and anticipated or demonstrated metabolic needs. FIG. 1 depicts a chart showing the RDA for various life stage groups.

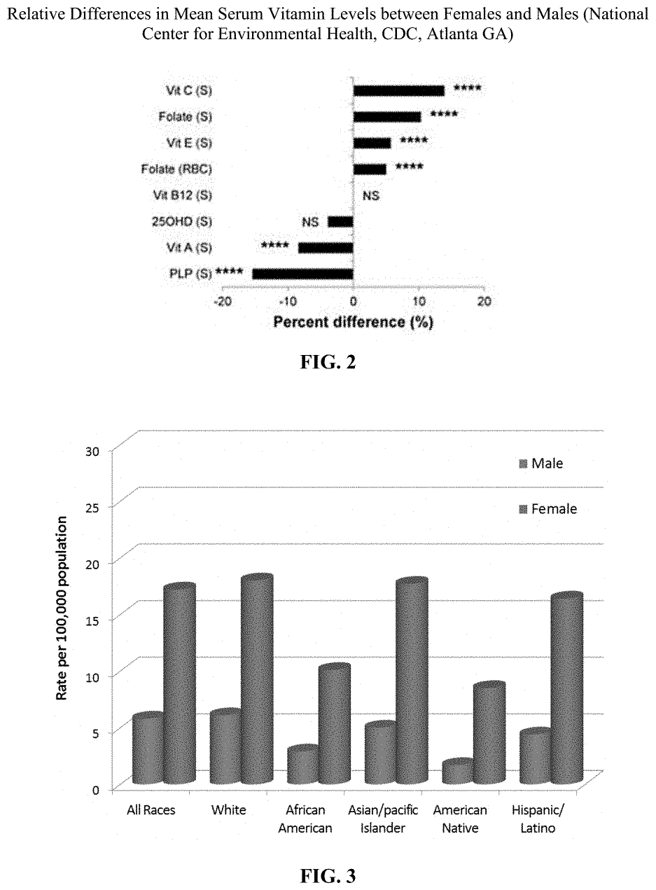

The Center for Disease Control (CDC) issued a report on biochemical indicators of diet and nutrition and looked at trends in vitamin levels by sex and found that when compared to males, females had lower vitamin A and vitamin B6 (PLP), but higher levels of vitamin C, folate, and vitamin E. This is further depicted in FIG. 2.

Differences in blood vitamin levels have also been found in response to food access and eating patterns with more fruit and vegetable-based diets yielding higher blood levels of an array of important nutrients as well as improved health outcomes. More impoverished populations with less access to a variety of healthy foods show lower blood vitamin level patterns and poorer health outcomes. In public health circles, it has long been accepted that “where you live” has a powerful impact on life expectancy even within a single county with differences of ten years within less than 20 miles. Impoverished urban areas with limited access to fresh fruit and vegetables as well as increased environmental toxins will require different supplementation than newer suburban neighborhoods with fewer adverse exposures.

On the other hand, micronutrient-fortified foods (cereal, bread, pasta, etc.) in the U.S. enjoy almost universal use. Because of the thoughtful inclusion of these micronutrients, many typical vitamin deficiencies are now almost non-existent. For example, thiamine (vitamin B1), riboflavin (vitamin B2), pantothenic acid (vitamin B5), and others have extensive replacement and therefore protect against significant population deficiencies. Accordingly, in many embodiments, an individualized micronutrient supplement, as described herein, does not need to contain these micronutrients.

Biotin deficiency is also very rare and happens in association with total or near total intravenous feeding without biotin supplementation. Accordingly, the micronutrient supplements disclosed herein can contain reduced amounts of biotin, or even no biotin at all, in certain embodiments.

Selenium is of fundamental importance to health and its deficiency has been linked to an array to poor health outcomes including impaired immune function, brain health, fertility issues, thyroid health, cancer and cardiovascular disease. Selenium unfortunately has a “U-shaped” pattern of benefit and detriment. Those with low selenium show the benefit of replacement but those with normal or high levels show adverse outcomes that may out-weight benefit. Some studies have shown poor outcomes associated with selenium supplementation and these occurrences make the addition to a micronutrient complicated. Selenium replacement supports thyroid health and increased thyroid cancer is seen in Asian, White and Hispanic Latino female populations as depicted in FIG. 3. Accordingly, micronutrient supplements described herein can include selenium for women of Asian, Hispanic/Latino, and European origin.

As can be appreciated, the practice of adding a number of non-vitamin trace elements to micronutrient supplements is prevalent but the rationale is far from evidence-based. Accordingly, the micronutrient supplements disclosed herein do not generally include manganese, iron, copper, or molybdenum absent a specific individualized need for such trace elements as there is no evidence of significant deficiencies of these trace elements in the general population.

Manganese is an essential trace element that is required for the activity of several enzymes but can also be toxic when ingested in large amounts. There are only a few scattered cases of manganese deficiency in the medical literature. Given the heterogeneity of the U.S. food supply with added fortification, manganese deficiency is atypical.

Both iron deficiency and iron excess can lead to impaired health status. Achieving optimal health outcomes requires a delicate balancing act so that iron intakes are neither too low nor too high for red blood cell formation and immune function. Iron replacement also gives a “U-shaped” risk curve. Replacement across a population is too risky for widespread supplementation and should only be included in specific individualized micronutrient supplements when necessary.

Copper is an essential trace metal that is required for several important biological processes, however, an excess of copper can be toxic to cells. Therefore, copper replacement should only be included in specific individualized micronutrient supplements when necessary.

Molybdenum deficiency is very rare and also happens in association with total or near total intravenous feeding without molybdenum supplementation. Replacement in a micronutrient supplement is only rarely needed.

Additionally, the amount of each vitamin necessary for a certain population can also vary by age or gender. For example, men tend to require more vitamin replacement due to size and muscle mass differences. Slowed renal and liver metabolism associated with age can also dictate a decreased dose of some minerals (potassium and magnesium to an extent). Research has confirmed that magnesium deficiency is a major driver of cardiovascular disease and been labeled a public health crisis.

Based on observations such as these, a number of micronutrient supplements are described herein that account for the differences in one or more of sex, age, height/weight (e.g., body mass index (“BMI”), pregnancy/lactation status, race/ethnicity, living environment, family income, family size, educational status and achievement, personal health history, family health history, eating status, and dietary inventory, as well as subcategories therein.

For example, differences in race and ethnicity can include whether a person is of African, Asian or Pacific Islander, European, Hispanic or Latino, or a combination thereof, descent. For living environment, the micronutrient supplement can account for whether an individual lives in a home, an apartment, an urban area, a suburban area, or a rural area, or some other factor such as metropolitan area, state, or zip code. Differences in personal health history and family health history can include cardiovascular health differences (e.g., whether someone has/had hypertension, stroke, heart attack, congestive heart failure, etc.), endocrine health differences (e.g., whether someone has/had diabetes, thyroid issues, etc.), renal diseases (e.g., dialysis, infections, etc.), cancers (e.g., lung, breast, colon, prostate, pancreas, etc.), bariatric or digestive track surgeries or alterations, any inherited disorders (e.g., hemoglobinopathies, etc.), bone health or fractures, smoking or nicotine replacement, alcohol consumption and amount, drug use (e.g., marijuana, cocaine, etc.), hormone replacement, anemia history, or depression history. Eating status differences can include whether an individual is vegan, vegetarian, pescatarian, an omnivore, etc. Finally, the micronutrient supplements can, in certain embodiments, be modified based on the dietary inventory of an individual such as whether, how often, and how much, they eat grains such as breads, cereals, rice, and/or pasta, whether they eat certain fruits and vegetables, whether they eat certain nuts and grains, and whether they eat certain meats.

Based on one or more of these characteristics, tailored micronutrient supplements have been formulated. As will be further described herein, certain micronutrient supplements have been developed for particular races and ethnicities. As can be appreciated, such micronutrient supplements are already individualized more than conventional micronutrient supplements.

African Americans and Populations of African Descent

With the added burden of having the worst health outcomes of any racial group in the United States, having a positive impact on the health of African Americans can be a significant contribution to society in terms of improved quality of life, enhanced productivity, and overall substantial health cost savings.

The most consistent and pronounced vitamin deficiency in African Americans in the literature relates to vitamin D. The recommended dietary allowance suggests 15 mcg/day (600 IU/day) while the literature suggests that African Americans need three to four times that amount daily (2000 IU/day based on population average levels). Low vitamin D levels have been associated with an increased risk for a number of diseases including: cancers of the breast, ovaries, prostate and colon, decreased asthma stability, and decreased cardiovascular and renal health. Accordingly, a micronutrient supplement tailored for African Americans and other high melanin content skin types can include substantially more vitamin D than the amount of vitamin D included in conventional micronutrient supplements.

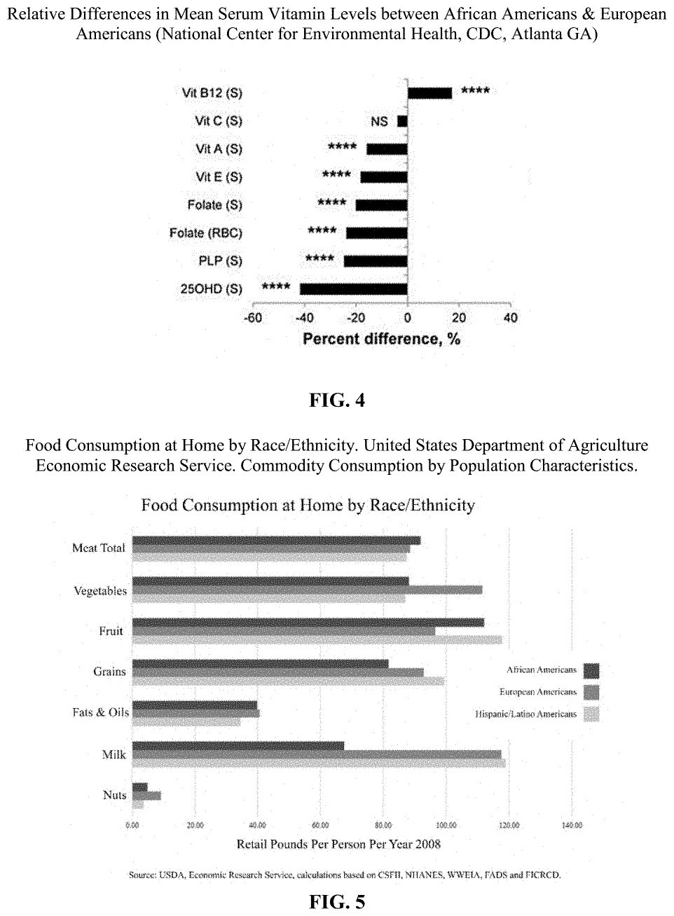

Population data also shows consistent deficiencies in folate, vitamin A, vitamin E, and vitamin B6 in African Americans. The Center for Disease Control (CDC) issued a report on biochemical indicators of diet and nutrition and looked at trends in vitamin levels by race and found that when compared to non-Hispanic whites, African Americans had lower vitamin A, folate, vitamin E, vitamin B6 (PLP), and vitamin D as depicted in FIG. 4. Their vitamin B12 levels were significantly higher than non-Hispanic whites. There is a risk that if folate is given to people with undiagnosed vitamin B12 deficiency it may lead to permanent neurological damage. Consequently, folate and vitamin B12 are best replaced in concert despite population-wide data showing a surplus of vitamin B12. The micronutrient supplements for African Americans described herein can include targeted replacement of each of these vitamins at appropriate levels.

As can be appreciated, higher vitamin C levels are inversely related to blood lead levels due to the vitamin's ability to inhibit intestinal absorption of lead as well as its ability to promote urinary excretion of lead. Increased poverty is directly related to increased lead exposure, and African Americans are disproportionately poor and live in poor urban (and rural) areas. Environmentalists confirm that urban air, soil, and water tend to hold comparably higher lead levels due to a history of industrial presence in cities and their proximity to neighborhoods disproportionately populated with African Americans. In certain embodiments, the micronutrient supplements described herein can have increased amounts of Vitamin C to increase the health of individuals living in urban and historically impoverished areas.

As can be appreciated, Vitamin C also has an array of benefits related to immunity and decreased atherosclerosis. Accordingly, in certain embodiments, the vitamin C dose for older African Americans can be comparatively higher.

Dietary patterns also inform suggestions for micronutrient replacement. The United States Department of Agriculture Economic Research Service has compiled “Commodity Consumption by Population Characteristics” data and they show differences in food consumption by race/ethnicity (depicted in FIG. 5). Overall, African Americans eat more meat (poultry, pork, and fish) and substantially less vegetables, grains, nuts, and milk than white Americans of European ancestry. Milk, for example, is an important source of calcium, vitamin B12, riboflavin, and phosphorus. Because of the high incidence of lactose intolerance in African Americans (75 percent of African Americans are lactose intolerant compared to 20 percent for white Americans of European descent), the nutritional benefits of milk evade the vast majority of African Americans. The decreased grain and nut consumption robs the consumer of various micronutrients including vitamin B6, vitamin E, thiamine, calcium, folate, niacin, magnesium, and zinc. The overall decreased consumption of vegetables, grains, and nuts deprives African Americans of a number of nutritional benefits that require compensatory replacement for optimal health. Accordingly, the micronutrient supplements disclosed herein can compensate for these dietary patterns.

Most consumers assume that micronutrient replacement in almost any quantity is safe and likely beneficial. But when combining the associations of some micronutrient supplements with adverse outcomes (increased cancer for example), the doubly increased risk in specific populations makes replacement a contraindication. Therefore, certain vitamins and minerals associated with negative outcomes can, and should, be proactively omitted from formulations based on race and ethnicity population outcomes.

Vitamin E supplements, for example, have been associated with increased “all-cause” mortality (cancers, cardiovascular, and more). African Americans have the highest mortality rate of any racial group in the United States and therefore should not be placed on a supplement that further worsens this condition. Vitamin E has been shown to increase the risk for prostate cancer in all male populations. Given African American males' significant increased prostate cancer incidence, aggressiveness, and mortality, the risk of providing any amount of vitamin E in a supplement for this population far out-weighs any potential benefit. FIG. 6 illustrates the significant increased death rate for the five top cancers for African Americans and the remarkable disparity between colon, breast and particularly prostate cancer mortality by race. Calcium supplementation in older African American men was significantly associated with increased prostate cancer but the same was not true for white American men of European descent. Therefore, an ideal micronutrient supplement for African American males would reduce or omit calcium except to the extent calcium is needed as a binder. β-Carotene supplementation was shown to be associated with increased lung cancer and gastric cancer across populations. Accordingly, certain micronutrient supplements disclosed herein can omit one or more of Vitamin E, calcium, and β-carotene.

Vitamin K is critical for normal blood clotting but African Americans have an increased propensity to form adverse blood clots after surgery and associated with strokes and other embolisms, therefore additional vitamin K in this population would be contraindicated. As such, in certain embodiments, micronutrient supplements disclosed herein can omit vitamin K.

There are vitamins and minerals that provide glucose stability to people with diabetes. According to the National Institute of Health, African Americans are twice as likely to develop diabetes than white Americans of European descent, and first line treatment involves metformin for half. Metformin can lead to folic acid and vitamin B12 deficiencies. Diabetes stability helps to evade the related end-organ adverse events including cardiovascular, renal, and neurological problems. Magnesium has been shown to improve diabetes control and stabilize endothelial function (which supports cardiovascular health) and certain micronutrient supplements disclosed herein can include magnesium.

Thiamine (vitamin B1) is disproportionately excreted in the presence of diuretics. African Americans are preferentially placed on diuretics when diagnosed with hypertension and/or congestive heart failure due to resounding outcomes data that show significantly increased efficacy in these populations. Seventy-five percent of African American adults age 55 or older meet the criteria for hypertension. Proactively replacing thiamine at a higher dose than other populations not given diuretics proactively also makes sense. With the increased diuretics use also comes comparatively more potassium excretion. Combine that added potassium excretion due to diuretics to data that show African Americans also have the lowest dietary potassium intake, and adding potassium to the supplement is both safe and sensible. Accordingly, the micronutrient supplements disclosed herein can include additional thiamine and potassium in certain embodiments.

Potassium has additionally shown benefits in cardiac arrhythmia stability, blood pressure control, and electrolyte balance. There has also been data that suggests African Americans have lower potassium levels overall which could be related to the increased incidence of type 2 diabetes and helpful with the stability of cardiovascular diseases.

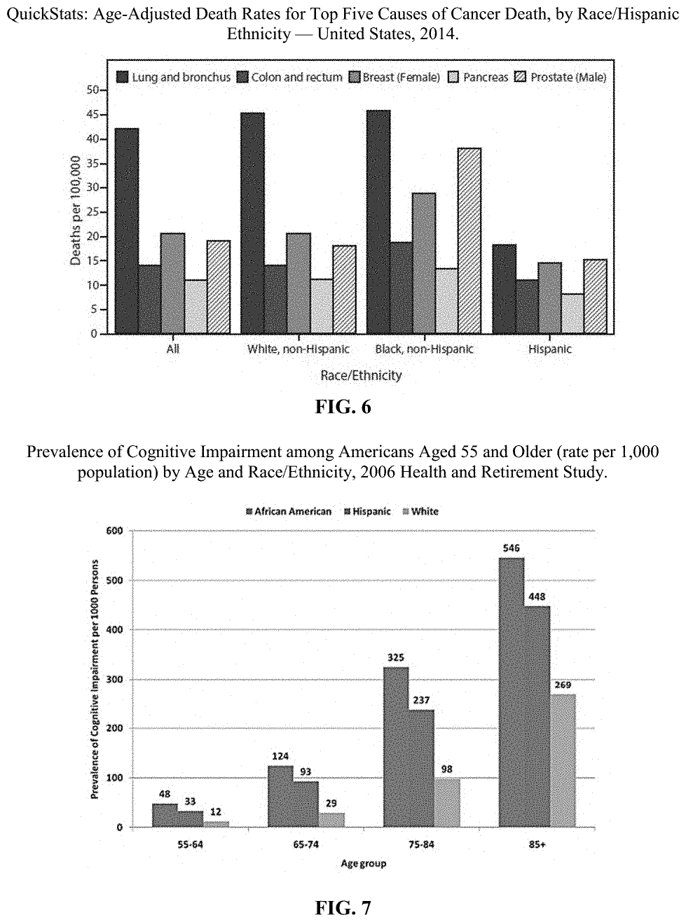

Thiamine also has significance relating to cognitive health because of its relation to serotonin secretion. Given African Americans (and Hispanic or Latino Americans) increased Alzheimer's Dementia in older adults, as depicted in FIG. 7, (who also have higher diuretic use), replacing thiamine losses is essential.

Additionally, thiamine, has a number of additional “groups at risk” for deficiency. For example, females have a higher risk for thiamine deficiency, and the risk increases with age. Accordingly, females will consistently need a higher dose of thiamine than males. Up to a third of older adults have thiamine deficiency as well so a formulation for more senior adults should also have a higher replacement dose. People with diabetes, which occurs 60% more often in African Americans compared to Whites, are also at increased risk for thiamine deficiency. Scientists have also seen a connection with thiamine deficiency and Alzheimer's disease. For example, thiamine deficiency produces oxidative stress in neurons, death of neurons, loss of memory, plaque formation, and changes in glucose metabolism all markers of Alzheimer's disease. Finally, there is also a connection between low thiamine levels and increased heart failure, which also occurs more in older adults, particularly in African Americans. Micronutrients disclosed herein can include relatively larger amounts of thiamine for such populations.

The increased renal disease in African Americans driven principally by diabetes shows the significant disparity between Native Americans and African Americans and other racial groups in the U.S. as depicted in FIG. 8. Having a positive impact on diabetes occurrence and control should narrow this difference.

Magnesium is the fourth most abundant mineral in the human body and plays a pivotal role in many of its functions, including muscle utility, energy metabolism, and cellular aging. Evidence has demonstrated associations between inadequate magnesium intake and increased risk of a host of adverse health outcomes, including cognitive decline, decreased muscle performance, brittle bones, type 2 diabetes, hypertension, and cancers of the breast, colon and others. African Americans (particularly at older ages) overwhelmingly have magnesium deficiency (>90 percent) with ongoing inadequate intake compared to white Americans of European descent as reported in a large study across multiple years. Adding magnesium to certain patient-centered micronutrient supplements will be beneficial in such embodiments.

Certain groups have a higher cardiovascular risk based on race, ethnicity or family history, and should receive more magnesium replacement. In hypertension, for example, magnesium status can have a direct effect upon the relaxation capability of vascular smooth muscle cells and the regulation of the cellular placement of other cations important to blood pressure. As a result, nutritional magnesium has both direct and indirect impacts on the regulation of blood pressure and therefore on the occurrence of hypertension. Many people with hypertension are treated with thiazide and loop diuretic medications that deplete the body of magnesium and potassium. Giving magnesium and potassium to patients with hypertension that are treated with diuretics reduces blood pressure and improves cardiovascular outcomes. Because of the high cardiovascular disease burden in African Americans, and particularly African American older men, the magnesium was purposefully NOT decreased based on age for the micronutrients disclosed herein.

Overall, African Americans have documented lower magnesium stores, less consumption as a population, higher hypertension and heart issues, and therefore a greater need. The same studies showed magnesium stores much better in Hispanic-Latino populations, therefore these populations would need less magnesium in both younger and older consumers.

Magnesium has also been shown to decrease the risk of breast cancer as well as slow its progression and/or recurrence. European American women have the highest breast cancer occurrence rate and would benefit from added magnesium. African American woman have a much higher mortality from breast cancer as well as an increased rate of recurrence and metastasis. For these reasons, a comparatively higher dose of magnesium is included in the younger formulation for African American women. Studies also show magnesium sufficiency associated with decreased colon cancer occurrence and progression.

Renal cell carcinoma also has a higher incidence in African Americans compared to white Americans of European descent and it has been suggested that higher dietary micronutrients including folate, thiamine, and vitamin C may have a protective effect.

African Americans also have a tendency toward zinc deficiency with an increased occurrence associated with increased poverty. Zinc has shown to be beneficial in boosting immunity to viruses including shortening the duration of colds. African Americans' disproportional infection rates and mortality during the COVID-19 pandemic demonstrates the potential benefit of including comparatively more zinc. Zinc has also shown benefit in sickle cell disease, reproductive health and depression. Sickle cell trait and disease is almost exclusively associated with being of African origin. Zinc should be a part of a micronutrient supplement for this population.

Chromium has promising data that it positively impacts diabetes control across populations. With African Americans having significantly higher risk for diabetes, adding chromium to the micronutrient supplement is potentially beneficial.

Due to its distinctive ability to neutralize free radicals, lycopene is believed to confer measurable protection against cancer, atherosclerosis, diabetes, and some inflammatory diseases. Evidence suggests that lycopene consumption is associated with decreased risk of various chronic diseases that disproportionately impact African Americans. In certain embodiments, the micronutrient supplements for African Americans can include lycopene.

Choline is an essential nutrient and a precursor to the neurotransmitter acetylcholine and has been shown to have a neuroprotective effect particularly with aging and the neurological damages associated with epilepsy and fetal alcohol syndrome. Choline should be a part of a micronutrient supplement for this population.

Based on these observations, a micronutrient supplement for African Americans can include, or exclude, the essential nutrients depicted in Table 1.

TABLE 1

| Vitamin | Benefit/Exclude |
|---|---|
| Vitamin A | Benefit |
| Vitamin B6 | Benefit |
| Vitamin B12 | Benefit |
| Vitamin C | Benefit |
| Vitamin D | Benefit |
| Vitamin E | Exclude |
| Vitamin K | Exclude |
| β-Carotene | Exclude |
| Calcium | Exclude |
| Choline | Benefit |
| Chromium | Benefit |
| Copper | Exclude |
| Folate | Benefit |
| Iron | Exclude |
| Lycopene | Benefit |
| Manganese | Exclude |
| Magnesium | Benefit |
| Molybdenum | Exclude |
| Niacin | Benefit |
| Potassium | Benefit |
| Riboflavin | Exclude |
| Selenium | Exclude |
| Thiamine | Benefit |
| Zinc | Benefit |

Populations with Asian and Pacific Islander Descent

Vitamin B12 deficiency within populations in the Asian Indian subcontinent have been solely attributed to dietary habits (generally the low consumption of meat). Genetic factors also influence vitamin status in these individuals. Indian populations have a high prevalence of vitamin B12 deficiency whether vegetarian or non-vegetarians when compared to populations of European descent. Vitamin B12 deficiency has also been associated with increased coronary artery disease in Indian populations whether vegetarian or not. The low vitamin B12 levels in Asian and Indian Americans has also been suggested to be associated with increased insulin resistance (and subsequent diabetes). Accordingly, certain micronutrient supplements for those of Asian and Pacific Islander descent can include additional Vitamin B12.

Intracerebral hemorrhage as a cause of stroke is at least two-fold more common to the individuals in the Asian population than European descent populations. Several studies suggest that the Asian population is more susceptible to bleeding events when treated with conventional antiplatelets, anticoagulants, and thrombolytic agents. The increased bleeding risk in the Asian population suggests that vitamin K can be beneficial in this population and can be included in certain micronutrient supplements disclosed herein.

A study looking at thiamine and depression in adults from China, age 50 to 70 years old, showed improved outcomes associated with higher thiamine levels. Thiamine diphosphate, the most bioactive form of thiamine, is a coenzyme in glucose metabolism crucial for the secretion of serotonin (which plays a vital role in depression) and plays an important role in nerve conduction. Another study found a direct correlation between blood thiamine metabolites and decreased Alzheimer's dementia. Because of the longer life expectancy of individuals in the Asian and Pacific Islander populations, increased dementia and depression associated with older age is an emerging problem.

β-carotene supplementation increases the risk for lung cancer and gastric cancer at doses of 20-30 mg per day. Asian populations have a much higher risk for gastric cancer therefore excluding β-carotene in order to further avoid gastric cancer is indicated.

Riboflavin (vitamin B2) deficiency is fairly rare in individuals of Asian and Pacific Islander descent due to “fortified foods” like cereal, bread, and milk. Significant riboflavin deficiencies are rare in this population and micronutrient supplements for this population can omit riboflavin.

Chromium has promising data that it positively impacts diabetes control across populations. With individuals of Asian and Pacific Islanders having a somewhat higher risk for diabetes, chromium can be included with certain micronutrient supplements for this population.

Due to its distinctive ability to neutralize free radicals, lycopene is believed to confer measurable protection against cancer, atherosclerosis, diabetes, and some inflammatory diseases. Evidence suggests that lycopene consumption is associated with decreased risk of various chronic diseases that impact individuals of Asian and Pacific Islander descent and, accordingly, micronutrient supplements for this population can include additional lycopene.

Selenium replacement supports thyroid health and increased thyroid disease is seen at increased levels in Asian female populations.

Based on these observations, a micronutrient supplement for individuals of Asian and Pacific Islander descent can include, or exclude, the essential nutrients depicted in Table 2.

TABLE 2

| Vitamin | Benefit/Exclude |
|---|---|
| Vitamin A | Benefit |
| Vitamin B6 | Benefit |
| Vitamin B12 | Benefit |
| Vitamin C | Benefit |
| Vitamin D | Benefit |
| Vitamin E | Exclude |
| Vitamin K | Benefit |
| β-Carotene | Exclude |
| Calcium | Exclude |
| Chromium | Benefit |
| Copper | Exclude |
| Folate | Benefit |
| Iron | Exclude |
| Lycopene | Benefit |
| Manganese | Exclude |
| Magnesium | Benefit |
| Molybdenum | Exclude |
| Niacin | Benefit |
| Potassium | Benefit |
| Riboflavin | Exclude |
| Selenium | Benefit (female) |
| Thiamine | Benefit |
| Zinc | Benefit |

Populations with European Descent

Fair-skinned people (those generally identified as white Americans of European descent) are a grouping of individuals having generally descended from Europe (although others from Western and Central Asia and North African are included). Because of their majority status in the United States, most of the research outcomes include white Americans of European descent as their largest participants. In short, our most complete information regarding micronutrient replacement is in this population.

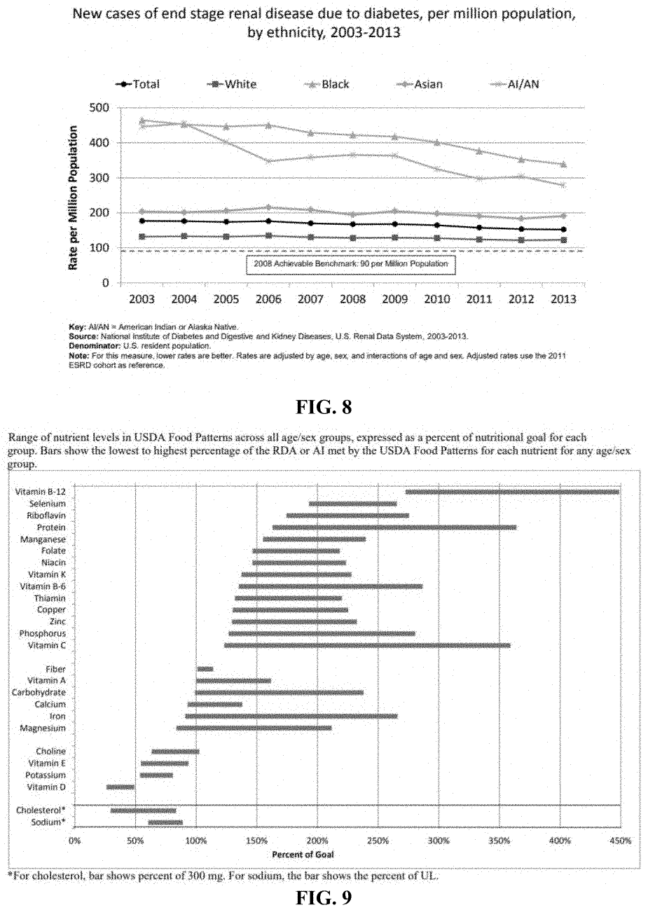

The U.S. Department of Health and Human Services put out a U.S. Department of Agriculture Scientific Report of the 2015 Dietary Guidelines Advisory Committee that analyzed the needs of Americans across populations but was weighted heavily with white Americans of European descent. FIG. 9 illustrates a range of nutrient levels and trends for under and over replacement. Like other groups, vitamin D deficiency is stark. Vitamin E, choline, and potassium show room for replacement. In certain embodiments, a micronutrient supplement for white Americans of European descent can include Vitamin D, Vitamin E, choline, and potassium.

Conversely, Vitamin B 12, folate, riboflavin and others show significant existing over-replacement across the population. Thiamine replacement seems adequate but emerging information related to a benefit related to mental health (because of its crucial role in the secretion of serotonin) makes its supplementation at a higher level advantageous. Niacin is adequately replaced however it has shown increased efficacy in lowering cholesterol levels in combination with “statin” medications as well as decreasing mouth and esophageal cancers which occur with great frequency in white Americans of European descent.

The addition of selenium to the micronutrient represents a case where the benefit differs by sex. The increased risk for thyroid cancer in white women of European descent is why selenium should be added to their formulation. Men do not have that increased risk therefore a micronutrient supplement for white male Americans of European descent, given their overall health status outcomes, should exclude selenium and vitamin E due to studies that suggest increased “all cause” mortality, as well as β-carotene and its increased risk for lung and prostate cancer.

Zinc is critical to immune function and its deficiency has been related to diseases such as rheumatoid arthritis, diabetes, atherosclerosis, impaired cognitive function, as well as age-related macular degeneration. These deficiencies tend to worsen with age. Besides the elderly, vegetarians or vegans and patients suffering from renal insufficiency or chronic diarrhea are also affected by zinc deficiency.

Based on these observations, a micronutrient supplement for white Americans of European descent can include, or exclude, the essential nutrients depicted in Table 3.

TABLE 3

| Vitamin | Benefit/Exclude |
|---|---|
| Vitamin A | Benefit |
| Vitamin B6 | Exclude |
| Vitamin B12 | Exclude |
| Vitamin C | Benefit |
| Vitamin D | Benefit |
| Vitamin E | Exclude |
| Vitamin K | Benefit |
| β-Carotene | Exclude |
| Calcium | Benefit |
| Choline | Benefit |
| Chromium | Benefit |
| Copper | Exclude |
| Folate | Exclude |
| Iron | Exclude |
| Lycopene | Benefit |
| Manganese | Exclude |
| Magnesium | Benefit |
| Molybdenum | Exclude |
| Niacin | Benefit |
| Potassium | Benefit |
| Riboflavin | Exclude |
| Selenium | Benefit (female) |
| Thiamine | Benefit |
| Zinc | Benefit |

Populations with Hispanic or Latino Descent

While broadly described as Hispanic or Latino, the people included in this category come from diverse regions including Mexican, Puerto Rican, Cuban, Central and South American as well as Southwestern Europe. As the largest minority subpopulation in the United States, Hispanics or Latinos also have unique micronutrient needs that should mirror population-wide deficiencies. In contrast to African Americans and populations of African descent, Hispanic or Latinos and Asian or Pacific Islanders have much less chronic disease burden and a significantly longer lifespan than both African- and European-descendant populations (African Americans have a life expectancy of 74.8 years; white Americans of European descent have a life expectancy of 78.5 years; Hispanic or Latino Americans have a life expectancy of 81.8 years; and Asian and Pacific Islander Americans have a life expectancy of 86.5 years). Some attribute the increased longevity to cultural differences and most specifically to dietary differences. There are however nuances in micronutrient replacement that can benefit this population as well.

For example, Hispanic or Latino populations have lower vitamin D levels and these low levels have been associated with increased breast cancer as well as increased asthma instability, but no connection to lung cancer or colon cancer could be found in the literature. There is good evidence that African Americans and Hispanic or Latino populations synthesize less vitamin D per unit of sun exposure than white Americans of European descent therefore supplementing more vitamin D in this population is advisable. Another study found a direct correlation between lower blood pressure and higher vitamin D levels in Hispanic or Latinos. The same study also found a positive correlation between higher vitamin D levels and higher HDL and lower LDL cholesterol levels (the more beneficial combination).

Folate deficiency is also more prevalent in Hispanic or Latino populations and folate deficiency also seems to increase asthma instability. Higher folate levels in Hispanic/Latino Americans were associated with a lower risk of breast cancer.

Vitamin B6 deficiency was found to be associated with increased depressive symptomatology in an older Latino population.

Riboflavin (vitamin B2) deficiency is fairly rare in Hispanic/Latino Americans due to “fortified foods” like cereal, bread, and milk.

Vitamin B12 deficiencies occur more in vegetarian populations and both Hispanic or Latinos and Asians have comparatively more widespread deficiencies.

Specific studies looking at the benefit of thiamine in Hispanic/Latinos are lacking however the lack of any connection between thiamine and bad outcomes in studied populations lend credence to its safety.

The Center for Disease Control (CDC) issued a report on biochemical indicators of diet and nutrition and looked at trends in vitamin levels by race and found that when compared to white Americans of European descent, Hispanic or Latino Americans had lower Vitamin E, folate, Vitamin A, vitamin B6 (PLP) and vitamin D. Their vitamin B12 levels were significantly higher than white Americans of European descent. Replacing micronutrients known to lower (and safe) in this population is beneficial and evidence-based.

Hispanic or Latino Americans (particularly at older ages) have magnesium deficiency (>84 percent) with ongoing comparable intake compared to white Americans of European descent as reported in a large study across multiple years. Adding magnesium to this patient centered micronutrient supplement will also be beneficial.

There is evidence that Hispanic or Latino Americans tend to have poorer diabetes outcomes and suboptimal self-monitoring of lipids, blood glucose, and blood pressure compared to non-Hispanic whites. Chromium has promising data that it positively impacts diabetes control acros s populations. With Hispanic or Latino Americans having comparatively higher risk for diabetes, certain micronutrient supplements described herein can include chromium.

Due to its distinctive ability to neutralize free radicals, lycopene is believed to confer measurable protection against cancer, atherosclerosis, diabetes, and some inflammatory diseases. Evidence suggests that lycopene consumption is associated with decreased risk of various chronic diseases that impact Hispanic or Latino Americans.

Based on these observations, a micronutrient supplement for Hispanic or Latino Americans can include, or exclude, the essential nutrients depicted in Table 4.

TABLE 4

| Vitamin | Benefit/Exclude |
|---|---|
| Vitamin A | Benefit |
| Vitamin B6 | Benefit |
| Vitamin B12 | Benefit |
| Vitamin C | Benefit |
| Vitamin D | Benefit |
| Vitamin E | Exclude |
| Vitamin K | Benefit |
| β-Carotene | Exclude |
| Calcium | Exclude |
| Choline | Benefit |
| Chromium | Benefit |
| Copper | Exclude |
| Folate | Benefit |
| Iron | Exclude |
| Lycopene | Benefit |
| Manganese | Exclude |
| Magnesium | Benefit |
| Molybdenum | Exclude |
| Niacin | Benefit |
| Potassium | Benefit |
| Riboflavin | Exclude |
| Selenium | Benefit (female) |
| Thiamine | Benefit |
| Zinc | Benefit |

### Interactive Person-Centered Micronutrient Supplementation

In certain embodiments, the micronutrient supplements disclosed herein can be further individualized. The prescriptive basic principle will coincide with the Latin foundational directive in medicine: “Primun Non Nocere” or “first, do no harm.” Micronutrients that have consistently shown the evidence of harm in a population or instance should be omitted proactively. The possibility of harm should be considered and weighed against the potential of benefit. Making the best decision will require more detailed information.

Such further micronutrient supplements can be individualized based on demographic information like sex, age, race/ethnicity, diet, environment, social situation, personal and family medical histories form the foundation on which an ideal micronutrient can be crafted.

In certain embodiments, this information can be entered into a digital database to analytically generate a micronutrient supplement that will both “do no harm” and supply maximal benefit. As can be appreciated, information used by the program can be updated as new information becomes available. As such, it is possible to constantly improve the formulation for a particular person based on changes in either research outcomes or personal/family history, for example. Additionally, many vitamin deficiencies are age-related, so as the person ages, the replacement will change.

Additionally, this digitally generated formulation can also be linked to a genetic profile or genetic ancestry website (such as 23andme.com) which can add further nuance to racial composition and make a potentially more “individualized” micronutrient supplement not just by using one race/ethnicity but by considering a number of races and subsequent risk factors.

### Formulations

Each of the active ingredients in the micronutrient supplements can be provided in any suitable form. For example, vitamins can be provided in the form of a salt or other compound as known in the art. In certain embodiments, Vitamin A can be provided as retinyl acetate; Vitamin C can be provided as ascorbic acid; Vitamin D can be provided as cholecalciferol; thiamine can be provided as thiamine mononitrate; niacin can be provided as niacinamide; Vitamin B6 can be provided as pyridoxine hydrochloride; folate can be provided as folic acid (and adjusted to provide a Dietary Folate Equivalent or DFE); Vitamin B12 can be provided as cyanocobalamin; magnesium can be provided as magnesium oxide; zinc can be provided as zinc oxide; chromium can be provided as chromium picolinate; and potassium can be provided as potassium chloride.

Generally, each active ingredient, can be included in a micronutrient supplement at an appropriate amount based on one or more of race, ethnicity, sex, age, height and weight, pregnancy/lactation status, living environment, family income, family size, educational status and achievement, personal health history, family health history, eating status, and dietary inventory. Suitable ranges for of each of the one or more vitamins or nutrients are depicted in Table 5.

TABLE 5

| Vitamin | Amount | |
|---|---|---|
| Vitamin A: | 0-3000 | mcg |
| Vitamin B6: | 30-100 | mg |
| Vitamin C: | 300-500 | mg |
| Vitamin D3: | 0.0125-0.05 | mg |
| Vitamin K: | 0-0.08 | mg |
| Vitamin B12: | 0.4-6 | mcg |
| Vitamin E: | 0-300 | IU |
| Folate: | 300 mcg-1 mg | |
| Magnesium: | 75-420 | mg |
| Thiamine: | 2-10 | mg |
| Riboflavin: | 0.4-3 | mg |
| Niacin: | 4-35 | mg |
| Zinc: | 3-40 | mg |
| Chromium: | 0-60 | mcg |
| Lycopene: | 0-20 | mg |
| Potassium: | 0-30 | mg |
| Choline | 0-20 | mg |
| Selenium: | 0-200 | mcg |
| Calcium: | 0-2000 | mg |

As can be appreciated, the micronutrient supplements described herein can be delivered in a number of different forms. For example, in certain embodiments, the supplements can comprise pill(s) or capsule(s) that can be taken quickly and efficiently on a regular basis (daily, with meals, etc.). As can be appreciated, pills and capsules can contain a number on inactive ingredients as known in the art such as dicalcium phosphate dehydrate, microcrystalline cellulose, stearic acid, silicon dioxide, croscarmellose sodium, magnesium stearate, and pharmaceutical glaze.

In other certain embodiments, the micronutrient supplements can be administered to individuals via food products. According to certain embodiment, the micronutrient supplements can be offered in the form of a health bar. In general, the micronutrient supplements can be embodied as comestibles having other form factors without departing from the scope of the invention. By way of illustration and not as a limitation, the constituents may be incorporated into a “smoothie” (fruit, vegetable, nut oil, or yogurt based), a frozen desert (e.g., ice cream or sorbet), and a beverage. For purposes of this application, these various forms will be referred to as “nutritional supplement products.”

### Examples

Table 6 depicts an exemplary multivitamin specifically formulated for males of African American descent. The multivitamin includes, as active ingredients, Vitamin A, Vitamin C, Vitamin D, thiamine, riboflavin, niacin, Vitamin B6, folate, Vitamin B12, choline, magnesium, zinc, chromium, potassium, and lycopene while omitting Vitamin E, Vitamin K, β-carotene, calcium, copper, iron, manganese, molybdenum, and selenium. The multivitamin further includes inactive ingredients including dicalcium phosphate dehydrate, microcrystalline cellulose, stearic acid, silicon dioxide, croscarmellose sodium, magnesium stearate, and pharmaceutical glaze.

TABLE 6

| Active Ingredients | Amount | |
|---|---|---|
| Vitamin A (retinyl acetate) | 1500 | mcg |
| Vitamin C (ascorbic acid) | 300 | mg |
| Vitamin D (cholecalciferol) | 50 | mcg |
| Thiamine (thiamine mononitrate) | 5 | mg |
| Riboflavin | 2 | mg |
| Niacin (niacinamide) | 16 | mg NE |
| Vitamin B6 (pyridoxine HCl) | 50 | mg |
| Folate (folic acid) | 1666.66 mcg (Dietary Folate | |
| | Equivalent (“DFE”): | |
| | 1000 mcg folic acid) | |
| Vitamin B12 (cyanocobalamin) | 6 | mcg |
| Choline (choline bitartrate) | 10 | mg |
| Magnesium (magnesium oxide) | 250 | mg |
| Zinc (zinc oxide) | 15 | mg |
| Chromium (chromium picolinate) | 60 | mcg |
| Potassium (potassium chloride) | 30 | mg |
| Lycopene | 1 | mg |

Table 7 depicts an exemplary multivitamin specifically formulated for females of African American descent. The multivitamin formulated for females of African American descent is similar to the multivitamin depicted in Table 6 for men but optimized for women by slightly modifying the amount of certain actives.

TABLE 7

| Active Ingredients | Amount | |
|---|---|---|
| Vitamin A (retinyl acetate) | 1200 | mcg |
| Vitamin C (ascorbic acid) | 200 | mg |
| Vitamin D (cholecalciferol) | 50 | mcg |
| Thiamine (thiamine mononitrate) | 5 | mg |
| Riboflavin | 2 | mg |
| Niacin (niacinamide) | 14 | mg NE |
| Vitamin B6 (pyridoxine HCl) | 50 | mg |
| Folate (folic acid) | 1666.66 mcg (Dietary Folate | |
| | Equivalent (“DFE”): | |
| | 1000 mcg folic acid) | |
| Vitamin B12 (cyanocobalamin) | 6 | mcg |
| Choline (choline bitartrate) | 10 | mg |
| Magnesium (magnesium oxide) | 320 | mg |
| Zinc (zinc oxide) | 15 | mg |
| Chromium (chromium picolinate) | 60 | mcg |
| Potassium (potassium chloride) | 20 | mg |
| Lycopene | 1 | mg |

Table 8 depicts an exemplary multivitamin specifically formulated for males of African American descent over the age of 50. The multivitamin is similar to the multivitamin for African American males under the age of 50 (Table 6) but with increased quantities of Vitamin C, thiamine, and zinc and reduced amounts of potassium to adjust for the differing needs of the older population.

TABLE 8

| Active Ingredients | Amount | |
|---|---|---|
| Vitamin A (retinyl acetate) | 1500 | mcg |
| Vitamin C (ascorbic acid) | 400 | mg |
| Vitamin D (cholecalciferol) | 50 | mcg |
| Thiamine (thiamine mononitrate) | 10 | mg |
| Riboflavin | 2 | mg |
| Niacin (niacinamide) | 16 | mg NE |
| Vitamin B6 (pyridoxine HCl) | 50 | mg |
| Folate (folic acid) | 1666.66 mcg (Dietary Folate | |
| | Equivalent (“DFE”): | |
| | 1000 mcg folic acid) | |
| Vitamin B12 (cyanocobalamin) | 6 | mcg |
| Choline (choline bitartrate) | 10 | mg |
| Magnesium (magnesium oxide) | 250 | mg |
| Zinc (zinc oxide) | 25 | mg |
| Chromium (chromium picolinate) | 60 | mcg |
| Potassium (potassium chloride) | 20 | mg |
| Lycopene | 1 | mg |

Table 9 depicts an exemplary multivitamin specifically formulated for females of African American descent over the age of 50. The multivitamin is similar to the multivitamin for African American females under the age of 50 (Table 7) but with increased quantities of Vitamin C, thiamine, and zinc and reduced amounts of magnesium, potassium, and lycopene to adjust for the differing needs of the older population.

TABLE 9

| Active Ingredients | Amount | |
|---|---|---|
| Vitamin A (retinyl acetate) | 1200 | mcg |
| Vitamin C (ascorbic acid) | 400 | mg |
| Vitamin D (cholecalciferol) | 50 | mcg |
| Thiamine (thiamine mononitrate) | 10 | mg |
| Riboflavin | 2 | mg |
| Niacin (niacinamide) | 14 | mg NE |
| Vitamin B6 (pyridoxine HCl) | 50 | mg |
| Folate (folic acid) | 1666.66 mcg (Dietary Folate | |
| | Equivalent (“DFE”): | |
| | 1000 mcg folic acid) | |
| Vitamin B12 (cyanocobalamin) | 6 | mcg |
| Choline (choline bitartrate) | 10 | mg |
| Magnesium (magnesium oxide) | 200 | mg |
| Zinc (zinc oxide) | 20 | mg |
| Chromium (chromium picolinate) | 60 | mcg |
| Potassium (potassium chloride) | 15 | mg |
| Lycopene | 0.8 | mg |

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

It should be understood that certain aspects, features, structures, or characteristics of the various embodiments can be interchanged in whole or in part. Reference to certain embodiments mean that a particular aspect, feature, structure, or characteristic described in connection with certain embodiments can be included in at least one embodiment and may be interchanged with certain other embodiments. The appearances of the phrase “in certain embodiments” in various places in specification are not necessarily all referring to the same embodiment, nor are certain embodiments necessarily mutually exclusive of other certain embodiments. It should also be understood that the steps of the methods set forth herein are not necessarily required to be performed in the orders described, and the order of the steps of such methods should be understood to be merely exemplary. Likewise, additional steps can be included in such methods, and certain steps may be omitted or combined, in methods consistent with certain embodiments.

What is claimed is:

1. A micronutrient supplement personalized for one or more personal characteristics of an individual, the personal characteristics comprising one or more of race, ethnicity, sex, age, height and weight, pregnancy/lactation status, living environment, family income, family size, educational status and achievement, personal health history, family health history, eating status, and dietary inventory.

2. The micronutrient supplement of claim 1 is personalized for people of African American descent.

3. The micronutrient supplement of claim 2 comprises reduced amounts of one or more of Vitamin E, Vitamin K, β-carotene, calcium, copper, iron, manganese, molybdenum, and selenium than a comparative micronutrient supplement.

4. The micronutrient supplement of claim 2 is substantially free of one or more of Vitamin E, Vitamin K, β-carotene, calcium, copper, iron, manganese, molybdenum, and selenium.

5. The micronutrient supplement of claim 2 comprises one or more of Vitamin A, Vitamin B6, Vitamin B12, Vitamin C, Vitamin D, choline, chromium, folate, lycopene, magnesium, niacin, potassium, magnesium, thiamine, riboflavin, and zinc.

6. The micronutrient supplement of claim 2 comprises:
about 500 mcg to about 2,500 mcg of Vitamin A;
about 100 mg to about 500 mg of Vitamin C;
about 15 mcg to about 90 mcg of Vitamin D;
about 0.5 mg to about 5 mg of riboflavin;
about 15 mg to about 100 mg of Vitamin B6;
about 800 mcg to about 3,500 mcg of folate;
about 2 mcg to about 12 mcg of Vitamin B12;
about 5 mg to about 15 mg of choline;
about 100 mg to about 500 mg of magnesium;
about 5 mg to about 30 mg of zinc;
about 30 mcg to about 120 mcg of chromium;
about 10 mg to about 50 mg of potassium; and
about 0.5 mg to about 1.5 mg of lycopene.

7. The micronutrient supplement of claim 2 is further personalized for one or more of sex, age, height and weight, pregnancy/lactation status, living environment, family income, family size, educational status and achievement, personal health history, family health history, eating status, and dietary inventory.

8. The micronutrient supplement of claim 1 is personalized for people of Asian and Pacific Islander descent.

9. The micronutrient supplement of claim 8 comprises reduced amounts of one or more of Vitamin E, β-carotene, calcium, copper, iron, manganese, molybdenum, and riboflavin than a comparative micronutrient supplement.

10. The micronutrient supplement of claim 8 is substantially free of one or more of Vitamin E, β-carotene, calcium, copper, iron, manganese, molybdenum, and riboflavin.

11. The micronutrient supplement of claim 8 comprises one or more of Vitamin A, Vitamin B6, Vitamin B12, Vitamin C, Vitamin D, Vitamin K, chromium, folate, lycopene, magnesium, niacin, potassium, thiamine, and zinc.

12. The micronutrient supplement of claim 1 is personalized for people of white American descent.

13. The micronutrient supplement of claim 12 comprises reduced amounts of one or more of Vitamin B6, Vitamin B12, Vitamin E, β-carotene, copper, folate, iron, manganese, molybdenum, and riboflavin than a comparative micronutrient supplement.

14. The micronutrient supplement of claim 12 is substantially free of one or more of Vitamin B6, Vitamin B12, β-carotene, copper, folate, iron, manganese, molybdenum, and riboflavin.

15. The micronutrient supplement of claim 12 comprises one or more of Vitamin A, Vitamin C, Vitamin D, Vitamin K, calcium, choline, chromium, lycopene, magnesium, niacin, potassium, thiamine, and zinc.

16. The micronutrient supplement of claim 1 is personalized for people of Hispanic or Latino descent.

17. The micronutrient supplement of claim 16 comprises reduced amounts of, Vitamin E, β-carotene, calcium, copper, iron, manganese, molybdenum, and riboflavin than a comparative micronutrient supplement.

18. The micronutrient supplement of claim 16 is substantially free of one or more of, Vitamin E, β-carotene, calcium, copper, iron, manganese, molybdenum and riboflavin.

19. The micronutrient supplement of claim 16 comprises Vitamin A, Vitamin B6, Vitamin B12, Vitamin C, Vitamin D, Vitamin K, choline, chromium, folate, lycopene, magnesium, niacin, potassium, and thiamine.

20. The micronutrient supplement of claim 1 is personalized for women of one or more of Asian and Pacific Islander descent, European descent, and Hispanic or Latino descent, and wherein the micronutrient supplement comprises selenium.
